(19) United States
(12) Patent Application Publication
(10) Pub. No.: US 2022/0415495 A1
(43) Pub. Date: Dec. 29, 2022

(54) SERVER FOR PROVIDING ASSESSMENT INFORMATION ON TOOTH BRUSHING

(71) Applicant: TEMARI, INC., Tokyo (JP)

(72) Inventor: Mari UTANO, Tokyo (JP)

(21) Appl. No.: 17/779,978

(22) PCT Filed: Nov. 26, 2020
(86) PCT No.: PCT/JP2020/044020

§ 371 Date: May 25, 2022

(30) Foreign Application Priority Data
Nov. 26, 2019 (JP) 2019-212848

(51) Int. Cl. G16H 40/63; G01P 15/18; G01C 19/00; A61C 17/22; A46B 15/00

(52) U.S. Cl. CPC G16H 40/63; G01P 15/18; G01C 19/00; A61C 17/221; A46B 15/0038; A46B 15/0002

(57) ABSTRACT

The purpose of this invention is to improve the health of users while effectively utilizing data obtained from information about tooth brushing. One embodiment of the present invention is a server connected via a network to a user terminal communicating with an instrument equipped with a sensor that detects predetermined measurement information on tooth brushing, and to an evaluator terminal that inputs predetermined evaluations, which receives measurement information on tooth brushing detected by the sensor, receives the measurement information on tooth brushing, receives the receiving habit information of a user who performs tooth brushing, and transmits the evaluation information based on the measurement information and the habit information to the user terminal.

### TECHNICAL FIELD

This invention relates to a server that provides evaluation information based on measurement information concerning tooth brushing.

### BACKGROUND TECHNOLOGY

Recently, the IoT (Internet of Things) has been gaining popularity. Internet of Things (IoT) services are becoming more and more widespread. With the spread of IoT (Internet of Things) services, services that collect information on tooth brushing and provide feedback to users in the fields of oral care and toothpaste prevention are also being provided.

For example, Patent Document 1 discloses a technology that collects detection data on the position and posture of a toothbrush, analyzes the data, and modifies the tooth brushing instruction based on the analysis results.

### PRIOR ART DOCUMENTS

### Patent Literature

[Patent Document No. 1] Japanese Patent Publication No. 2017-130-873

### SUMMARY OF THE INVENTION

### The Problem to be Solved by the Invention

However, the above-mentioned technology is related to an improvement of tooth brushing instruction by an instructor to each individual. On the other hand, oral care is not limited to the mere prevention of tooth decay, but is also related to other diseases and habits, and is linked to the health problems of society as a whole. Therefore, it is necessary to make effective use of the data obtained from toothbrushing information to promote the health of users in a wide range of areas.

The purpose of this invention is to improve the health of users by making effective use of data obtained from information on tooth brushing.

### Means to Solve the Problem

One embodiment of the present invention is a server connected via a network to a user terminal communicating with an apparatus equipped with a sensor that detects predetermined measurement information concerning tooth brushing, and to an evaluator terminal that inputs predetermined evaluations, wherein said server receives measurement information regarding tooth brushing detected by said sensor and receives habit information of a user who performs tooth brushing, and transmits evaluation information based on said measurement information and said habit information to said user terminal.

### The Effect of the Invention

According to the present invention, it is possible to improve the health of the user while effectively utilizing data obtained from information on tooth brushing.

### BRIEF DESCRIPTION OF THE DRAWINGS

FIG. 1: A diagram of the system according to the first embodiment of the present invention.

FIG. 2: An example of a functional block diagram of a server according to the first embodiment of the present invention.

FIG. 3: An example of a block diagram showing the details of the control unit of a server according to the first embodiment of the present invention.

FIG. 4: An example of a block diagram showing details of the storage of a server according to the first embodiment of the present invention.

FIG. 5: An example of a measurement information management table according to the first embodiment of the present invention.

FIG. 6: An example of an evaluation data management table according to the first embodiment of the present invention.

FIG. 7: An example of a user data management table according to the first embodiment of the present invention.

FIG. 8: An example of a flowchart showing an example of the evaluation information provision process by a server, according to the first embodiment of the present invention.

The following is a list and a description of the contents of the embodiments of the present invention. The server according to the embodiment of the invention has the following configuration.

### [Item 1]

A server connected via a network to a user terminal communicating with an instrument equipped with a sensor that detects predetermined measurement information on tooth brushing; and to an evaluator terminal that inputs a predetermined evaluation, and

wherein the server receives measurement information on tooth brushing detected by the sensor;

receives habit information of a user who performs tooth brushing; and

transmits evaluation information based on the measurement information and the habit information to the user terminal.

### [Item 2]

The server according to Item 1, wherein the measurement data includes information regarding at least one of the data and the time of tooth brushing, the time per brushing or the number of brushings, the speed or the angle of brushing, and the width of the brush.

### [Item 3]

The server according to Item 1, wherein the habit information includes one of the user's diet, meal times, waking, sleeping, and biometric information.

### [Item 4]

The server according to Item 1, wherein the evaluation information includes information regarding illness or lifestyle improvement.

### [Item 5]

The server according to Item 1, wherein the server receives the evaluation information from the evaluator terminal.

### First Embodiment

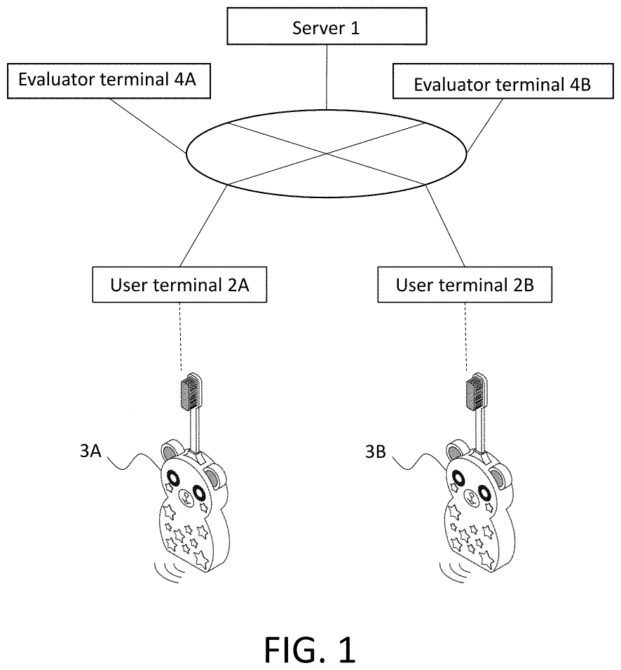

The processing by the server according to the first embodiment of the present invention will be described below with reference to the drawings. FIG. 1 is a configuration diagram of the system according to the first embodiment of the present invention.

As shown in FIG. 1, the system includes a server 1 that provides services, and a plurality of user terminals 2A, 2B, etc. that are connected to the server 1 via a network such as the Internet. The server 1 is also connected to one or more evaluator terminal servers 4A, 4B via a network. Furthermore, the user terminals 2A, 2B each have a short-range communication such as Bluetooth and communicate with the device (in this embodiment, a toothbrush holder) via a network such as the Internet.

The server 1 can provide the service to the user terminal 2 via the application. The user terminal 3 downloads an application from the server 1 or another server, executes this application, and accesses the server 1 via web page browsing software such as a browser to send and receive information to and from the server 1, making it possible to receive services

The user terminal 2 performs a short-range wireless communication with a sensor (not shown) such as an acceleration sensor or a gyro sensor attached to an instrument 3 (in this embodiment, a toothbrush holder, but another electric toothbrush or the like may be used). By doing so, it is possible to acquire predetermined measurement information regarding toothbrushing from the sensor.

The acceleration sensor is a sensor that detects acceleration in three axis directions (x-axis, y-axis, and z-axis directions) orthogonal to each other, and is provided in the instrument 3. The front-back direction of the toothbrush is defined as the X direction, the left-right direction is defined as the Y direction, and the up-down direction is defined as the Z direction. As a result, the server 1 can perform predetermined arithmetic processing based on the information obtained from the acceleration sensor and acquire information on the speed and position of the toothbrush. Further, the type of sensor is not limited to this, and any sensing device that can acquire information on movement, such as a gyro sensor and a motion sensor, can be adopted. The gyro sensor is a sensor that detects the angular velocity around the x-axis, y-axis, and z-axis, and the server 1 calculates the rotation speed based on the angular velocity by a predetermined arithmetic process and acquires information on the posture of the toothbrush.

Returning to FIG. 1, the user terminal 3 and the evaluator terminal 4 may be a general-purpose computer such as a workstation or a personal computer, or may be a smartphone, a tablet, a mobile terminal, or other information terminal. Here, the user terminal 3 is assumed to be used by a family user who brushes teeth at home, and the evaluator terminal 4 is assumed to be used by a specialist such as a dentist or a doctor, but the present invention is not limited to this.

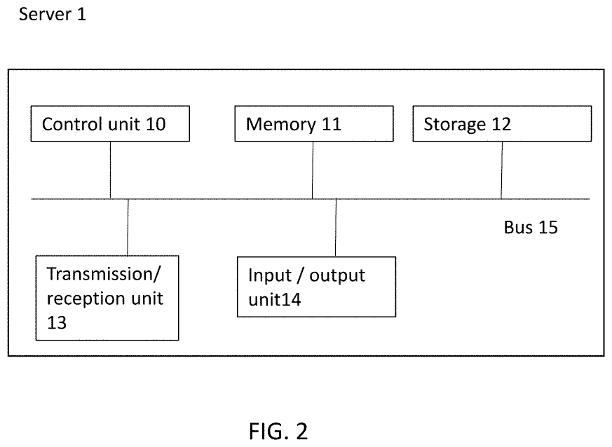

FIG. 2 is a functional block diagram of the server 1 according to the first embodiment of the present invention. The configuration shown in the figure is an example, and may have other configurations.

As shown, the server 1 is connected to a database (not shown) to form part of the system. The server 1 may be a general-purpose computer such as a workstation or a personal computer, or may be logically realized by cloud computing.

The server 1 includes at least a control unit 10, a memory 11, a storage 12, a transmission/ reception unit 13, an input/output unit 14, and the like, and these are electrically connected to each other through a bus 15.

The control unit 10 is an arithmetic unit that controls the operation of the entire server 1, controls the transmission and reception of data between each element, and performs information processing necessary for application execution and authentication processing. For example, the control unit 10 is a CPU (Central Processing Unit), and executes each information processing by executing a program or the like stored in the storage 12 and expanded in the memory 11. It should be noted that the function of the control unit 10 provided in the server 1 can be executed by the control unit (not shown) of the user terminal 2.

The memory 11 includes a main storage composed of a volatile storage device such as a DRAM (Dynamic Random Access Memory) and an auxiliary storage composed of a non-volatile storage device such as a flash memory or an HDD (Hard Disk Drive). The memory 11 is used as a work area or the like of the processor 10, and also stores a BIOS (Basic Input/Output System) executed when the server 1 is started, various setting information, and the like.

The storage 12 stores various programs such as application programs. A database (not shown) storing data used for each process may be built in the storage 12. Information stored in the storage 12 provided in the server 1 (for example, measurement information detected by the sensor of the instrument 3, user information including user habit information, evaluation information generated based on the measurement information, etc.) can also be executed by a storage unit (not shown) of the user terminal 2.

The transmission/reception unit 13 connects the server 1 to the network. The transmission/reception unit 13 may be provided with a short-range communication interface of Bluetooth (registered trademark) and BLE (Bluetooth Low Energy).

The input/output unit 14 is an information input device such as a keyboard and a mouse, and an output device such as a display.

The bus 15 is commonly connected to each of the above elements and transmits, for example, an address signal, a data signal, and various control signals.

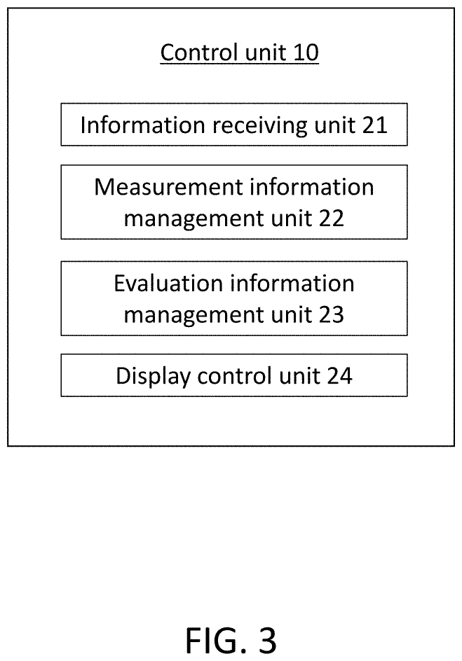

FIG. 3 is an example of a functional block diagram showing details of the control unit 10 of the server according to the first embodiment of the present invention. As described above, the control unit 10 executes an application program related to the service according to the present embodiment, and the module of the executed program is divided into several functional blocks as shown in the figure.

The information receiving unit 21 receives the measurement information, which is detected by the sensor provided in the instrument 3 and transmitted via the user terminal 3, via the transmitting/receiving unit 13 of the server 1. The received measurement information is stored in the measurement information storage unit 31 of the storage 12 built in the server 1. Alternatively, it can be stored in the storage built in the evaluator terminal 4 shown in FIG. 1.

The measurement information management unit 22 performs predetermined arithmetic processing based on the received measurement information, and stores the calculated information in the measurement information storage unit 31 as received measurement information or as updated measurement information.

The evaluation information management unit 23 stores the evaluation information received from the evaluator terminal 4 in the evaluation information storage unit 32 of the storage 12 built in the server 1. Further, the evaluation information storage unit 32 stores predetermined evaluation standard information for evaluating the measurement information, and the evaluation information management unit 23 evaluates the measurement information based on the evaluation standard information and generates the evaluation information. Then, it can be stored in the evaluation information storage unit 32. Alternatively, evaluation information is generated by machine learning using the measurement information about the tooth brushing of a plurality of users stored in the measurement information storage unit 32 and the evaluation information stored in the evaluation information storage unit 32 as teacher data. The generated evaluation information can also be stored in the evaluation information storage unit 32. Here, the evaluation information management unit 23 can also generate evaluation information based on the habit information and the like included in the user information 800 described later.

In addition, the control unit 10 has a display control unit 25 that generates data comprising a screen displayed on the display of the user terminal 3.

As described above, the measurement information related to the user's tooth brushing detected by the sensor provided in the instrument 3 is stored in the user terminal 2, and is built in the user terminal 2 together with the information such as the user's habit information. It is also possible to evaluate the information by a program and output the evaluation information to the user 3's display. As a result, even when the user terminal 2 is offline and is not connected to the server 1 via the network, the user terminal 2 can execute a predetermined process such as analysis and evaluation of the user's tooth brush.

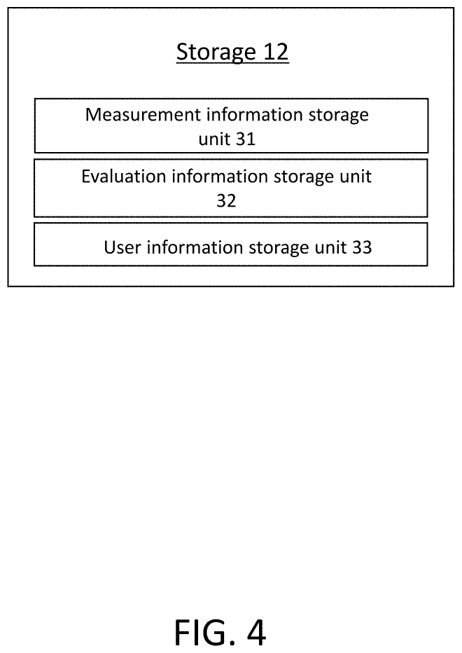

FIG. 4 is an example of a block diagram showing details of server storage according to the first embodiment of the present invention.

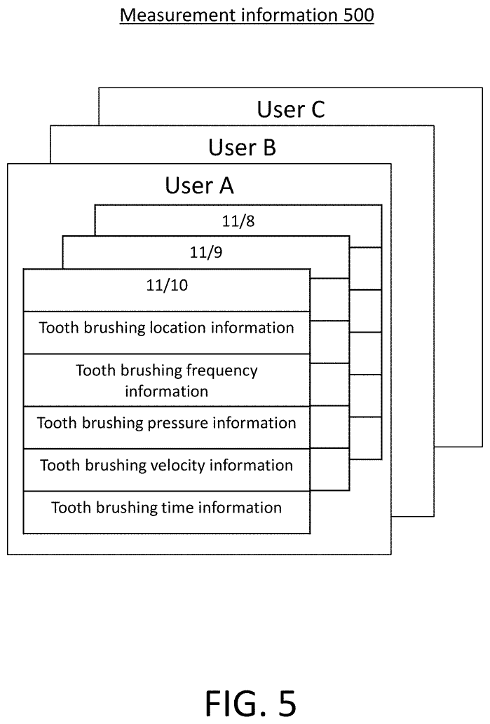

The measurement information storage unit 31 stores the measurement information 500 shown in FIG. 5. As shown in FIG. 5, the measurement information includes predetermined measurement information regarding the user's tooth brushing on a daily (or date and time) basis. For example, as shown in FIG. 5, information on the position of the toothbrush (eg, the width of a predetermined movement (reciprocating motion) of the user's toothbrush, the position of the toothbrush relative to a specific tooth/part, the posture, etc.), information on the number of times of brushing (eg, the number of predetermined movements (reciprocating movements, etc.) of the toothbrush per specific tooth/part or toothbrush), information on toothbrush pressure (eg, pressure on a specific tooth, average pressure per toothbrush) Etc.), information on toothbrushing speed (eg, average speed per toothbrushing, etc.), toothbrushing time information (eg, specific tooth/site or time required for one toothbrushing), etc. are included, but not limited to this.

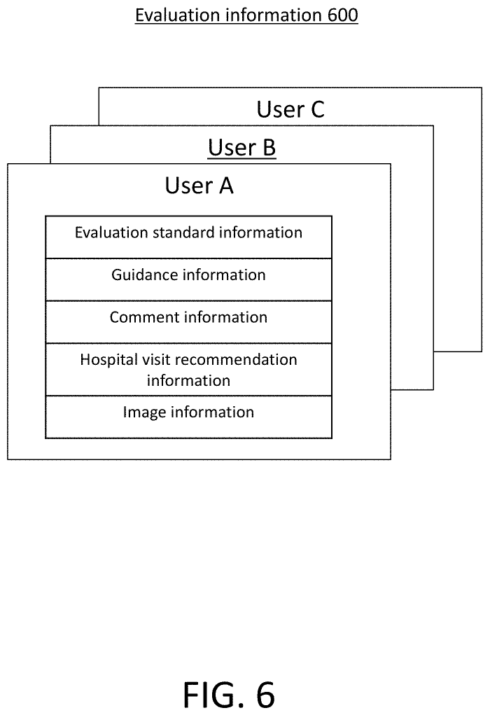

The evaluation information storage unit 32 stores the evaluation information 600 shown in FIG. 6. As shown in FIG. 6, as evaluation information, evaluation reference information (for example, correlation of data acquired from measurement information of all users (average tooth brushing time; average number of tooth brushing times; average pressure; average tooth brushing speed; average tooth brushing motion; tooth brushing time; speed, etc.) to dental caries and other diseases, and correlation of habit information to dental caries and other diseases), guidance information for users (eg, comparison between user's tooth brushing and standard information, tooth brushing scoring, pre-stored template improvement instruction comments for tooth brushing), comment information (eg, free text comments from evaluators, etc.), recommended visit time information (eg, information on when users are recommended to visit a dental clinic or hospital, information on the urgency of visits, etc.), and image information (eg, 2D or 3D animation display generated based on the measurement information of the user's tooth brush), but is not limited to this.

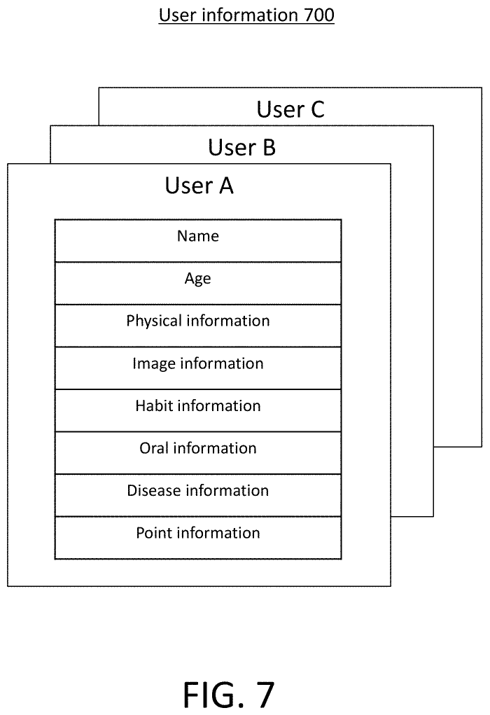

The user information storage unit 33 stores the user information 700 shown in FIG. 7. As shown in FIG. 7, as user information, age information (or date of birth) of the user (user who brushes teeth), physical information (eg height, weight, etc.), image information (eg, user's face photo, video of brushing teeth, user's X-ray photograph taken from the dental clinic or hospital where the user goes to the hospital, etc.), habit information (eg, meal date and time, meal content, number of meals, snacks, exercise, sleep time, etc.), oral information (eg, number of teeth, presence or absence of dental caries, jaw size, skeleton, etc.), disease information (eg medical history, surgical history, etc.), and point information held by the user is included, but not limited to this.

It should be noted that all or part of the functional blocks executed by the main control unit of the server can also be executed by the control unit (not shown) of the user terminal 3 depending on its nature. For example, by storing the various information in the storage unit of the user terminal 3 and having the control unit execute each of the above functions, it is possible to realize the function executed by the server 1 even in an offline environment. Further, as a user terminal, for example, by equipping a mirror (for example, a hand mirror) used by the user with an instrument (toothbrush holder or electric toothbrush) with a control unit and a storage unit, each function can be similarly realized

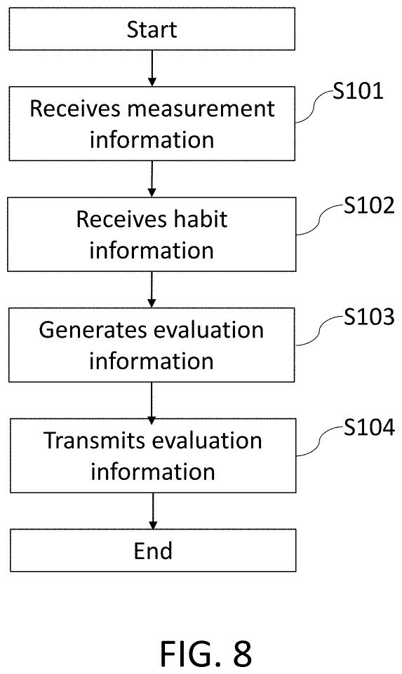

FIG. 8 is an example of a flowchart showing an example of evaluation information provision processing by the server. This process is realized, for example, by executing the program stored in the memory 11 of the server 1 in the control unit 10.

First, the information receiving unit 21 of the control unit 10 of the server 1 receives the measurement information measured by the sensor provided in the instrument from the user terminal 2 via the transmitting/receiving unit 13 (S101). The information receiving unit 21 stores the received measurement information in the measurement information storage unit 31 of the storage 12. Here, the measurement information management unit 22 of the control unit 10 can perform predetermined arithmetic processing based on the received measurement information, and store the calculated information as the measurement information in the measurement information storage unit 31. For example, if the measurement information received by the information receiver 21 is parameter information about the acceleration of the toothbrush obtained from the accelerometer, the measurement information management unit 32 can perform a predetermined integral process to calculate and store the measurement information as the toothbrush's speed information and/or position information.

Next, the information receiver 21 receives the habit information from the user terminal 2 via the transmitter/receiver 13 (S102). As an example of habit information, the user can input information about daily meals (e.g., contents (or images) of meals, date and time of meals, meal times, whether or not the user snacks, and date and time of snacking) via a predetermined user interface displayed via an application installed on the user terminal 2 is input, the habit information is transmitted to the server 1. The information receiving unit 21 stores the received habit information in the user information storage unit 33 of the storage 12.

Next, the evaluation information management section 23 of the control unit 10 receives evaluation information from the evaluator terminal 4 (S104). As a preliminary process, the measurement information management unit 22 sends the measurement information and habit information to the evaluator terminal 4, and the evaluator terminal 4 generates evaluation information based on the received measurement information and habit information. In generating the evaluation information, in the evaluation information management unit 23 of server 1, based on the evaluation standard information stored in the evaluation information storage unit 32 (e.g., correlation of data from the measurement information of all users (average tooth brushing time, average tooth brushing frequency, average pressure, average tooth brushing velocity, average tooth brushing motion, tooth brushing time, velocity, motion) to tooth decay and other diseases, and correlation of habit information to tooth decay and other diseases, etc.), the user's tooth brushing can be evaluated and evaluation information about tooth brushing and/or health can be generated. For example, the evaluation information management unit 23 can generate evaluation information about tooth brushing, such as “take more time to brush” with a comparative display that the user's tooth brushing time is below the average tooth brushing time. Alternatively, the evaluation information management unit 23 can focus on the short brushing time and generate evaluation information regarding health and/or lifestyle habits, such as “get up earlier” or “get more sleep”. The evaluator (dentist, doctor, etc.) using the evaluator terminal 4 can then input additional comments from an expert perspective via the user interface displayed via an application installed on the evaluator terminal 4. The expert comment is transmitted to the server 1, and the evaluation information management unit 23 can store the received expert comment as comment information in the evaluation information storage unit 32. The evaluation information can be stored in the evaluation information storage unit 32. The evaluation standard information can also be stored in the evaluator's terminal 4, and an evaluator comment can be added after the evaluation information is generated in advance at the evaluator's terminal 4. The specialist can also perform the evaluation by linking the above measurement information, etc., with the user's medical interview and diagnostic information held by the dentist or hospital. In addition, the above measurement information and health information (lifestyle information, medical interview information and/or diagnosis information) held by a single or multiple dental clinics and/or hospitals or obtained by the user can be accumulated, managed and evaluated by the health platform, and then health information (lifestyle or diagnosis—related evaluation information) can also be provided to the user. The evaluation information includes not only the measurement information of the user's tooth brushing at one time, but also the evaluation of the user's tooth brushing over a predetermined period of time such as the past day, week, month, etc. based on the past measurement information. In this case, the measurement information on tooth brushing of other users can also be used by referring to the measurement information accumulated for a predetermined period of time. In addition, scoring, ranking, etc. of the user's tooth brushing can be generated based on the evaluation criteria information as other evaluation information. Here, evaluation information based on measurement and/or habit information of multiple users residing in the area where the user resides can be displayed, or scoring and ranking (as national or regional averages) can be displayed as evaluation information. By providing information on other users, each user can be motivated to improve their teeth brushing and their lifestyle. Also, by working on the same game while brushing teeth together with other users, each user can be motivated to improve their tooth brushing. The evaluation information management unit 23 can also evaluate the urgency of the user to visit the hospital based on the tooth brushing measurement and/or habit information, decide whether or not the user needs to visit the hospital, and notify the user to that effect. The system can then present the outpatient hours of a given dental clinic and hospital (e.g., a dental clinic or hospital in the area and/or a specific dentist or doctor), or process the user to make an appointment. In this case, the entity operating server 1 may receive a portion of the consultation fee paid by the user to the dental hospital or hospitals when the user actually visits and receives medical treatment. For example, the remuneration obtained from tooth brushing instruction provided by a dentist to a patient based on measurement and/or habit information and the products provided in relation to this instruction can be accumulated as medical points. Similarly, the remuneration obtained from tooth brushing instruction provided to users at welfare facilities and products provided in connection therewith can be accumulated as welfare points. As described above, the total or average of the medical and welfare points of multiple users residing in the user's area of residence can also be provided to each user. Here, data such as measurement information of adult users, especially senior users, can also be collected, a welfare platform can be built, said data can be stored as welfare data, and welfare scores based on said data can be recorded and managed in said platform.

The display control unit 24 then processes the images to be displayed on the user board-face displayed on the user terminal 2 based on the evaluation information (S104). Here, the evaluation information management unit 23 can award points based on the user's evaluation of the tooth brushing. The user can accumulate the points awarded as prevention points and exchange them for goods/services provided by stores or service providers with which the administrator managing server 1 is affiliated. In addition, to encourage users to actively send measurement information, points can be awarded each time they enter predetermined information or brush their teeth using the device and send measurement information. The server 1 can also total the number of preventive points for each user based on predetermined conditions, for example, by totaling the total or average number of preventive points by municipality based on the user's area of residence, or by hospital or dental clinic based on the hospital or dental clinic that the user visits, or by or the average value can be tabulated and used as a reference as an evaluation indicator for the municipality or hospital/dental clinic. Alternatively, points can be added by the user brushing his/her teeth or based on the user's evaluation of brushing his/her teeth as points (insurance points) to be referred to by insurance companies as the basis for calculation when estimating the insurance amount. In addition to points based on the above measurement information, points can also be awarded based on dental visit information and other information (hospital visit-related information, lifestyle-related information) as insurance points. In this way, the insurance company can also calculate and provide an estimate of the insurance product to be offered to the user based on the above measurement information and/or lifestyle-related information.

This means that, according to this embodiment, not only measurement information on tooth brushing obtained from the user, but also habit information can be utilized to implement and provide more accurate evaluation of tooth brushing to the user.

As described above, the method of providing evaluation information by the server has been described. The evaluator (dentist or doctor) using the above evaluation terminal 4 can make comments or send alerts to the user based on the measurement and/or habit information and/or evaluation information received from the server 1. The dentist or doctor can then provide medical care to the patient user while referring to the measurement information. The dentist or doctor can also perform remote treatment such as online treatment. By utilizing the measurement information provided by the system, the dentist or doctor can send patients to the dentist or doctor, and the dentist or doctor can provide more accurate medical care by referring to the information. In addition, a dentist, etc. can provide advice to the user on the user's oral condition and tooth brushing, etc. at a remote location away from the user, based on the intraoral images taken by the user himself/herself with the user terminal 2, etc. and the above measurement information, etc. Here, the above intraoral image is made into a 3D model, and based on measurement information, etc., the areas where teeth are being brushed and areas where teeth are not being brushed are visualized, and the visualized 3D model image can be shared with the user while providing advice.

In addition to measurement information, server 1 can also acquire information about the mother-child notebook from user terminal 2 or other sources. For example, the server 1 acquires, as information related to the mother-child handbook, information related to the birth and childcare of the user's mother when the user is a child, as well as the user's growth records such as height and weight; analyzes the correlation between the separately obtained measurement information and the user's mother's birth and child-rearing records or the user's height and weight, and; for example, generates and transmits evaluation information on the user's tooth brushing so that the user's growth curve can be maintained within an appropriate range. Also, in conjunction with the user's health checkup information, evaluation information can be generated based on the above-mentioned acquired measurement information and transmitted to the user.

The server 1 can also acquire weather information as well as measurement information from the user terminal 2 or other sources. In particular, by acquiring weather information for the area where the user is located, the correlation between the separately acquired measurement information and weather conditions (temperature, humidity, weather) can be analyzed to generate and transmit, for example, evaluation information on the user's tooth brushing according to weather condition.

The server 1 can also acquire medical interview information as well as measurement information from the user terminal 2 or another source (in this example, the evaluator's terminal). For example, by acquiring the contents of a medical interview (information on cough, throat swelling, sneezing, medications being taken, diarrhea, etc.) conducted by hospital personnel to the user, the correlation between the separately acquired measurement information and the contents of the medical interview can be analyzed to generate and transmit evaluation information on the user's tooth brushing according to his/her physical condition. In addition to the interview information, the correlation between the above-obtained measurement information and the diagnosis information can also be analyzed, and evaluation information can be generated and transmitted based on the diagnosis information managed by the dentist or doctor, as described above.

The above-described embodiments are merely examples to facilitate understanding of the invention and are not intended to be construed as limiting the invention. It goes without saying that the invention may be changed and improved without departing from its purpose, and that the invention includes its equivalents.

### DESCRIPTION OF REFERENCE NUMERALS

1 Server

2 User terminal

3 Instrument

4 Evaluator terminal

1. A server connected via a network to a user terminal communicating with an instrument equipped with a sensor that detects predetermined measurement information regarding tooth brushing; and to an evaluator terminal that inputs a predetermined evaluation,
wherein the server receives the measurement information on tooth brushing detected by the sensor; and
receives habit information of a lifestyle of a user who performs tooth brushing; and
transmits evaluation information based on the measurement information and the habit information to the user terminal.

2. The server according to claim 1, wherein the measurement data includes information regarding either the date and time of tooth brushing, the duration per brushing or the frequency, the velocity or angle of tooth brushing, and the width of tooth brushing.

3. The server according to claim 1, wherein the habit information includes one of the following: the user's diet, meal time, waking up, sleeping, and biometric information.

4. The server according to claim 1, wherein the evaluation information includes the information about one of disease or life improvement.

5. The server according to claim 1, wherein the evaluation information is received from the evaluator's terminal.
